(19) United States
(12) Patent Application Publication
(10) Pub. No.: US 2025/0197937 A1
(43) Pub. Date: Jun. 19, 2025

(54) SYSTEM AND METHOD FOR HIGH THROUGHPUT EVALUATION OF CHEMICAL MODIFICATIONS OF NUCLEIC ACID MOLECULES

(71) Applicant: ELEVEN THERAPEUTICS LTD, Ramat Gan (IL)

(72) Inventors: Yaniv ERLICH, London (GB); Omer ZIV; Arun TANPURE, Cambridge (GB)

(21) Appl. No.: 18/846,704

(22) PCT Filed: Mar. 13, 2023
(86) PCT No.: PCT/EP2023/056298

§ 371 Date: Sep. 13, 2024

Related U.S. Application Data
(60) Provisional application No. 63/319,350, filed on Mar. 13, 2022.

(51) Int. Cl. C12Q 1/6876; G16B 40/20

(52) U.S. Cl. CPC C12Q 1/6876; G16B 40/20; C12Q 2600/178

(57) ABSTRACT

This disclosure relates to method and system for evaluating chemical modifications of oligonucleotide molecules including, for example, siRNA molecules, utilizing a second oligonucleotide associated with the modified oligonucleotide, wherein the sequence of the second oligonucleotide is indicative of the chemical identity of the modified oligonucleotide, and the chemical modification profile of the modified oligonucleotide molecule is associated with its functional readouts.

### FIELD OF THE INVENTION

Disclosed herein are systems and methods for high throughput identification and evaluation of chemical alterations of various nucleic acid molecules, including, for example, siRNA molecules.

### BACKGROUND OF THE INVENTION

Oligonucleotide technologies (ONT) is an emerging class of programmable therapies that consists of a short polymer of nucleic acids. This class includes siRNA (short interfering RNA), miRNA (microRNA), guide RNA (gRNA), ASO (antisense oligos), GapmeR, and others. All of these oligonucleotides bind to a target and usually recruit an enzyme, such as Ago2 in the case of siRNA or RNase H in the case of GapmeR, to facilitate their therapeutic effect.

ONT can provide a potent and specific method for modulating genetic targets in human cells, but translating these technologies into a therapeutically potent modality have had limited success in maturing into clinically validated drugs, thus far. Clinical and research efforts to avoid toxicity and achieve desirable safety and efficacy profiles focus on target selection, sequence selection, trigger design, chemical formulation, and delivery mechanisms.

A major challenge is the ability to sustain a continuous gene silencing effect without requiring repeated dosing. However, RNA molecules quickly wane even in non-proliferating cells, thereby countering the vision of gene therapy with a long-lasting effect. For example, in their naked form, non-integrating double-stranded 19-25nt long siRNA molecules display poor pharmacokinetics properties as they are subjected to degradation by RNA nucleases and undergo autohydrolysis due to self-cleavage.

This hurdle that prevents a durable effect has been successfully addressed in several clinically ONT by using chemically modified nucleotides that extended their effect to last for over a few months.

However, chemical optimization of oligonucleotides is currently an empirical, laborious, bespoke process of slow R&D cycles of trial-and-error exercise in a massive search space that leads to years of development and high failure rate. Furthermore, the optimization necessitates a balance between efficacy and stability, but to date, the field has yet to elucidate the structure-activity relationship (SAR) with respect to chemical modifications of ONT.

Current screening methods for chemically modified ONT rely on low-throughput column-based synthesis platforms coupled with poorly scaled standard reporter assays.

Therefore, there is a need for improved methods and systems to allow high throughput identification and evaluation of the effect of chemical modifications on the activity of nucleic acid molecules, such as, siRNA, miRNA, and ASO molecules.

### SUMMARY OF THE INVENTION

According to some embodiments, provided herein are system, platform and method for high throughput identification and evaluation of chemical modifications of various nucleic acid molecules. According to some embodiments, provided herein a system (also referred to herein as “DeepSii” and “DeepSii platform”) enabling a massively parallel method of evaluating and predicting the effect of chemical modifications on the duration of the effect of various oligonucleotide molecules, ranging from siRNAs to other nucleic acid-based therapeutics, such as ASOs (antisense oligonucleotides), CRISPR/Cas9 gRNAs, and aptamers. DeepSii lays out a platform to generate, assay, and analyze massive amounts of data in order to rationally design long lasting ONT therapies with properties similar to non-integrating DNA based gene therapies.

Advantageously, opposed to currently used screening methods for chemically modified ONT that rely on low-throughput column-based synthesis platforms coupled to poorly scaled standard reporter assays, the systems and methods disclosed herein enable order of magnitude greater throughput and cost-effectiveness evaluation of long-lasting ONT, and further prediction of optimal combinations of chemical modifications.

According to some embodiments, these beneficial features are facilitated utilizing a massively-parallel approach, which brings together a combinatorial chemistry design for simultaneous synthesis of large collection of modified oligonucleotide molecules and corresponding DNA barcodes encoding their chemical pattern, with a high-throughput design of a functional assay for evaluating the chemically modified nucleic acids molecules (such as RNAi molecules), and further optionally with a deep learning and artificial intelligence (AI) system tools for further analyses and suggestions regarding the optimal durability of these potential therapeutic molecules. Furthermore, the disclosed high-throughput systems and methods for evaluating and suggesting chemically modified oligonucleotides can immensely expedite the discovery of hyper-potent molecules without compromising efficacy for stability. Hence, in some exemplary embodiments, it may be implemented for improving siRNA effect in the lungs, which could benefit treatments for a wide range of related diseases.

According to some embodiments, provided is a method for evaluating chemical modifications of oligonucleotide molecules, the method comprising:
(a) obtaining a plurality of beads each of the plurality of beads comprising a first oligonucleotide molecule, and a second oligonucleotide molecule, wherein the first oligonucleotide molecule is attached to the beads via a cleavable linker, wherein the first oligonucleotide molecule of each of the plurality of beads has a same sequence with a different chemical modification profile, and wherein a sequence of the second oligonucleotide molecule is indicative of the chemical modification profile of the first oligonucleotide molecule,
(b) juxtaposing cells to the beads, wherein the first oligonucleotide is intended for altering a biological function/activity of the cells;
(c) releasing the first oligonucleotide molecule from the beads, thereby allowing its uptake by the cells;
(d) sorting the cells based on functional readouts,
(e) determining the sequence of the second oligonucleotide molecule on the sorted cells or beads;
(f) decoding the chemical modification profile of the first oligonucleotide molecule, based on the determined sequence of the second oligonucleotide molecule; and
(g) associating the chemical modification profile of the first oligonucleotide molecule with its functional readouts.

According to some embodiments, the method further comprises selecting oligonucleotides with an optimal chemical modification profile, based on their functional readouts. According to some embodiments, the optimal chemical modification profile provides a sustained/desired change in the function of the oligonucleotide and/or an enhanced change in the function of the oligonucleotide, relative to a same oligonucleotide without the optimal chemical modification profile.

According to some embodiments, the first and second oligonucleotides may be independently DNA or RNA molecules. According to some embodiments, the first oligonucleotide molecule is an mRNA molecule. According to some embodiments, the first oligonucleotide molecule is part of an mRNA molecule that will be ligated post-synthesis to create a full-length mRNA. According to some embodiments, the first oligonucleotide molecule is an siRNA molecule. According to some embodiments, the siRNA comprises a hairpin that folds on itself. According to some embodiments, the siRNA is a single stranded siRNA. According to some embodiment, the single stranded siRNA is annealed on beads to the complementary siRNA strand

According to some embodiments, the first oligonucleotide molecule is a gRNA molecule for a CRISPR system, an ASO molecule, a gRNA molecule for ADAR, an mRNA, an LNA, or an aptamer or any combination thereof. Each possibility is a separate embodiment.

According to some embodiments, the first oligonucleotide molecule is connected to the beads via a cleavable linker. According to some embodiments, the cleavable linker is a photocleavable linker.

According to some embodiments, the second oligonucleotide is DNA. According to some embodiments, the second oligonucleotide comprises PCR primers annealing sites.

According to some embodiments, the method further comprises a step of conjugating a small molecule to the first oligonucleotide molecule, or to both the first and the second oligonucleotide molecules; the small molecule facilitating and/or enhancing cell entry. In a preferred embodiment the small molecule facilitates and/or enhances receptor mediated endocytosis. Small molecules useful for facilitating the uptake of the oligonucleotide are known by the skilled person in the art. According to some embodiments, the small molecule is selected from the group of Vitamin E, Cholesterol, GalNac, Cholesterol-PEG, Cholesteryl-triethylene glycol (Cholesterol-TEG), Lithocolic-oleyl, Lauryl, Myristoyl, Palmitoyl, Steroyl, Docosanyl, Oleoyl, Linoleoyl, or any combination thereof. In a preferred embodiment, said small molecule is Vitamin E, Cholesterol-TEG, and/or GalNac. Each possibility is a separate embodiment.

According to some embodiments, the juxtaposing of the cells to the beads comprises growing/proliferating cells directly on the beads. According to some embodiments, the proliferation of the cells is controllable. According to some embodiments, the cells are subject to contact inhibition. According to some embodiments, the sorting of the cells comprises sorting the cells together with the beads on which they are grown.

According to some embodiments, the juxtaposing of the cells to the beads comprises encapsulating the cells and the beads into single nano compartments, such that each nano compartment receives no more than one bead and at least one cell, and wherein releasing the first oligonucleotide comprises releasing it into the nano compartment. In a preferred embodiment said nano compartments are droplets. According to some embodiments, the droplets are agarose droplets. According to some embodiments, the method further comprises releasing at least one first oligonucleotide molecule from the bead and into the droplet, thereby allowing its uptake by the cells. According to some embodiments, the method further comprises releasing the second oligonucleotide from the bead and into the droplet, thereby allowing its uptake by the cells. According to some embodiments, the sorting of the cells comprises recovering the cells from the droplet. According to some embodiments, the sorting of the cells comprises sorting the cells, after their recovery from the droplet.

According to some embodiments, the cells express at least one endogenous and/or exogenous reporter gene. Preferably, cells express at least one exogenous reporter gene.

According to some embodiments, step (c) of the method comprises releasing the first and the second oligonucleotide from the beads.

According to some embodiments, the method further comprises applying a machine learning module, wherein the applying comprises providing a first input regarding a combination of chemical modification profile on each of the first oligonucleotide, and a second input regarding the sequence of the first oligonucleotide molecule. According to some embodiments, the output of the machine learning module is a combination of chemical modification associated with optimal first oligonucleotide function. According to some embodiments, the applying of the machine learning module comprises applying a feedback generative adversarial network.

According to some embodiments, provided is a computational platform for high-throughput analysis of chemical modifications of oligonucleotides, the platform comprising a processor configured to:
(a) obtain data indicative of a chemical modification pattern of a plurality of beads each of the beads comprising an oligonucleotide molecule having a same sequence with a different chemical modification profile, wherein the oligonucleotide is capable of affecting the functional read-out of its biological activity
(b) obtain data indicative of the functional read-out of the biological activity of the oligonucleotide; and
(c) apply a machine learning algorithm to:
(d) determine the chemical modification pattern, based on the data indicative thereof; and
(e) associate the chemical modification profile of the oligonucleotide molecule with the functional readout of the reporter gene.

In some embodiments, the data according to step (a) is obtainable or obtained according to the method of the present invention.

According to some embodiments, the processor is further configured to identify oligonucleotide sequences having an optimal chemical modification profile, based on the association.

According to some embodiments, the processor is further configured to apply a same or different machine learning algorithm to derive a structure-activity-relationship capable of predicting an optimal chemical modification profile of an oligonucleotide associated with one or more desired characteristics of the oligonucleotide, wherein the one or more characteristics is selected from stability, efficacy, durability, or any combination thereof.

According to some embodiments, the machine learning module (=machine learning method) or algorithm is selected from the group of:
Generative adversarial networks (using methods such as conditional GANs, self-supervised GANs, GANs with a Generative Adversarial Classifier)
Evolutionary algorithms (using methods such a Wright-Fisher model, a top-k model, ADALEAD, CMA-ES)
AI-guided directed evolution (using methods such as CbAS and DbAS)
Bayesian optimization (using methods such as BO-EVO)
Reinforcement learning (using methods such as DQN and PPO)
Discrete optimization methods (using methods such as basin-hopping, particle swarm optimization, and simulated annealing)
   or combinations thereof.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

As referred to herein, the terms “polynucleotide molecules”, “oligonucleotide”, “polynucleotide”, “nucleic acid” and “nucleotide” sequences may interchangeably be used. The terms are directed to polymers of deoxyribonucleotides (DNA), ribonucleotides (RNA), and modified forms thereof in the form of a separate fragment or as a component of a larger construct, linear or branched, single stranded (ss), double stranded (ds), triple stranded (ts), or hybrids thereof. Accordingly, as used herein, the terms “polynucleotide molecules”, “oligonucleotide”, “polynucleotide”, “nucleic acid” and “nucleotide” sequences are meant to refer to both DNA and RNA molecules. The terms further include oligonucleotides composed of naturally occurring bases, sugars, and covalent inter nucleoside linkages, as well as oligonucleotides having non-naturally occurring portions, which function similarly to respective naturally occurring portions. As used herein, nucleotides (A, G, C or T/U) and nucleotide sequences are marked in lowercase letters (a, g, c or t/u).

As used herein, a “nucleotide” includes a nitrogenous base, a sugar molecule, and a phosphate group. A nucleic acid may include naturally occurring nucleosides (e.g., adenosine, thymidine, guanosine, cytidine, uridine, deoxyadenosine, deoxythymidine, deoxyguanosine, and deoxycytidine), nucleoside analogues (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, locked nucleic acids, arabinose, and hexose), and artificial bases (e.g. dNaM or d5SICS).

As used herein, the term “chemical modification” refers to nucleotides that include changes in the phosphate group, the base, the sugar, the attachment of a small molecule, or combinations thereof.

As used herein, the terms “pattern” and “profile” of chemical modifications may interchangeably be used and may refer to the combination of modifications of an oligonucleotide molecule.

According to some exemplified embodiments, the chemical modifications of the sugar are 2′-deoxy-2′-Fluoro (2′-F) and 2′-O-Methyl (2′-OMe). Other non-limiting examples of chemical modifications to the sugar include: glycol nucleic acid, 2′-methoxyethyl (2′-MOE), locked nucleic acid (LNA), and unlocked nucleic acid (UNA).

In some embodiments, the chemical modification of the phosphate group is phosphorothioate (PS). Non-limiting examples of chemical modifications to the phosphate group include: vinylphosphonate, peptide-bond, methylphosphonate, and phosphorodithioate.

In some embodiments, the chemical modification of the base is N6-methyladenosine. Non-limiting examples of chemical modifications to the base include: pseudouridine, 5-nitroindole, and 5-methylcytidine.

In some embodiments, the chemical modification by attachment of a small molecule is N-acetylgalactosamine (GalNAc) to the 5′-end. Non-limiting examples of chemical modifications using a small molecule attachment include Vitamin E, Cholesterol, or Folic acid to the 5′ end.

As used herein, the term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g. by DNA replication or transcription of DNA or RNA, respectively). DNA and RNA can also be chemically synthesized. RNA can be post-transcriptionally modified. The terms “target mRNA” and “target transcript” are synonymous as used herein.

As used herein, the term “RNA interference” (“RNAi”) refers to selective intracellular degradation of RNA (also referred to as gene silencing). As used herein a RNAi molecule may collectively refer to small interfering RNAs and short hairpin RNA.

As used herein, the term “RNAi trigger”, refers to siRNA, microRNA, or shRNA or any other RNA molecule that triggers that RNAi machinery.

As used herein, the term “small interfering RNA” (“siRNA”), also referred to in the art as “short interfering RNAs,” refers to an RNA (or RNA analogue) comprising between about 10-60 or 15-25 nucleotides (or nucleotide analogues) that is capable of directing or mediating RNA interference. Generally, as used herein the term “siRNA” refers to double stranded siRNA (as compared to single stranded or antisense RNA). In certain embodiments, the 3′ end of the RNAi molecules may include additional nucleotides that create an overhang, such as “TT”.

As used herein, the term “short hairpin RNA” (“shRNA”) refers to an siRNA (or siRNA analogue) precursor that is folded into a hairpin structure and contains a single stranded portion of at least one nucleotide (a “loop”), e.g., an RNA molecule that contains at least two complementary portions hybridized or capable of hybridizing to form a double-stranded (duplex) structure sufficiently long to mediate RNAi (as described for siRNA duplexes), and at least one single-stranded portion, typically between approximately 1 and 10 nucleotides in length that forms a loop connecting the regions of the shRNA that form the duplex portion. The duplex portion may, but typically does not, contain one or more mismatches and/or one or more bulges consisting of one or more unpaired nucleotides in either or both strands. Without wishing to be bound by theory, shRNAs are thought to be processed into siRNAs by the conserved cellular RNAi machinery. shRNAs are capable of inhibiting expression of a target transcript that is complementary to a portion of the shRNA (referred to as the antisense or guide strand of the shRNA). In general, the features of the duplex formed between the guide strand of the shRNA and a target transcript are similar to those of the duplex formed between the guide strand of an siRNA and a target transcript. In certain embodiments of the invention the 5′ end of an shRNA has a phosphate group while in other embodiments it does not. In certain embodiments of the invention the 3′ end of an shRNA has a hydroxyl group.

According to some embodiments, the RNA molecule is a single stranded RNA molecule, such as but not limited to a single stranded siRNA (e.g. guide strand only). According to some embodiments, the single stranded RNA is intended for hybridization with a complementary strand e.g. within the target cell.

An RNAi-inducing entity is considered to be targeted to a target transcript for the purposes described herein if (1) the agent comprises a strand that is substantially complementary to the target transcript over 15-29 nucleotides, e.g., 15, more preferably at least about 17, yet more preferably at least about 18 or 19 to about 21-23 or 24-29 nucleotides. For example, in various embodiments of the invention the agent comprises a strand that has at least about 70%, preferably at least about 80%, 84%, 89%, 90%, 91%, 92%, 93%, 94%, 95%, 96%, 97%, 98%, 99%, or 100% precise sequence complementarity with the target transcript over a window of evaluation between 15-29 nucleotides in length, e.g., over a window of evaluation of at least 15, more preferably at least about 17, yet more preferably at least about 18 or 19 to about 21-23 or 24-29 nucleotides in length; or (2) one strand of the RNAi agent hybridizes to the target transcript under stringent conditions for hybridization of small (<50 nucleotide) RNA molecules in vitro and/or under conditions typically found within the cytoplasm or nucleus of mammalian cells.

As used herein the term “target” “target gene” and “target site” may be used interchangeably and may be any gene or transcript, the regulation of which is desired. According to some embodiments, the target site may be a human gene or transcript. According to some embodiments, the target site may be a gene or transcript of a pathogen, such as but not limited to a virus.

As used herein, the term “reporter gene” refers to a gene encoding a reporter protein (for example a fluorescent reporter protein, such as but not limited to Venus, GFP, RFP, mCherry, split-GFP, split-mCherry, etc.), the activity/expression of which can be monitored/measured in a functional assay. The reporter gene can also be a gene that gives proliferation/survival/resistance advantage or disadvantage to the cells. The reporter gene may be any gene that allows the monitoring/measurement in a functional assay. In certain embodiments a reporter gene is a gene expressed (giving rise to RNA transcripts) in a cell for which RNA or protein output can be measures qualitatively and/or quantitatively. In some embodiments reporter genes can be exogenous genes or endogenous genes.

In the various embodiments, the level of activity/expression of the reporter gene may be affected by the function or activity of the first oligonucleotide molecule, thereby enabling the extraction of functional readouts in a functional assay.

As used herein, the term “functional readouts” refers to the measure of function or activity of the first oligonucleotide molecule as indicated by a reporter gene in a functional assay.

As used herein, the term “functional assay” may refer to the use of a reporter gene that scores the long-term efficacy of chemical modifications of the first oligonucleotide molecule.

As used herein, the term “function” or “activity” may interchangeably be used.

As used herein, the term “long term” refers to a period of at least 30 minutes, at least one hour, at least one day, at least one week, at least two weeks, at least one month, at least three months, at least one year.

As used herein, the terms “massively parallel”, “high throughput” and “large scale” may interchangeably be used and relate to the simultaneous synthesis, screening, and/or analyses of at least 50, at least 100, at least 500, at least 1000 or at least 10,000 oligonucleotide modifications using the DeepSii platform.

As used herein, the term “guide strand” refers to the strand of the siRNA which is incorporated into the RNA-induced silencing complex (RISC) and which causes degradation of the transcript to which it pairs.

As used herein, the term “the passenger strand” is the strand of the siRNA complementary to the guide strand and which is degraded when the two strands separate.

As used herein, the term “complementary” refers to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence, the nucleotides form a complementary base pair, and the nucleic acids are complementary at that position. One of ordinary skill in the art will appreciate that the nucleic acids are aligned in antiparallel orientation (i.e., one nucleic acid is in 5′ to 3′ orientation while the other is in 3′ to 5′ orientation). A degree of complementarity of two nucleic acids or portions thereof may be evaluated by determining the total number of nucleotides in both strands that form complementary base pairs as a percentage of the total number of nucleotides over a window of evaluation when the two nucleic acids or portions thereof are aligned in antiparallel orientation for maximum complementarity. According to some embodiments, if the window of evaluation is 15-16 nucleotides long, substantially complementary nucleic acids may have 0-3 mismatches within the window; if the window is 17 nucleotides long, substantially complementary nucleic acids may have 0-4 mismatches within the window; if the window is 18 nucleotides long, substantially complementary nucleic acids may have may contain 0-5 mismatches within the window; if the window is 19 nucleotides long, substantially complementary nucleic acids may contain 0-6 mismatches within the window. In certain embodiments the mismatches are not at continuous positions. In certain embodiments the window contains no stretch of mismatches longer than two nucleotides in length. In preferred embodiments a window of evaluation of 15-19 nucleotides contains 0-1 mismatch (preferably 0), and a window of evaluation of 20-29 nucleotides contains 0-2 mismatches (preferably 0-1, more preferably 0). In addition, according to some embodiments, the positions of the mismatches within the RNAi triggers play an important role. For example, in miRNA mismatches outside the second to seventh positions from the 5′ end of the RNAi trigger (known as “the seed region”) may be tolerated in RNAi triggers that mimic or harness microRNA like repression.

As used herein, the term “nano compartment” refers to an enclosed space comprising at least one bead and at least one cell according to the invention which is separated from other nano compartments. Separation in this context can be achieved by compartmentalization of beads and cells in separated volumes such as droplets preventing mixing of multiple nano compartments. In some instances, nano compartments may be separated from each other only temporary and can be combined at a desired time.

As used herein, the term “droplet” refers to any amount of a liquid, solution, emulsion, foam, gel, suspension and/or hydrogel with a volume of about 1 fL to 10 ml. A microdroplet refers to a droplet with a volume of about 50 pL to 500 nl.

As used herein, the terms “approximately” or “about” in reference to a number are generally taken to include numbers that fall within a range of 5% or in the range of 1% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

As used herein, the singular forms “a,” “an” and “the” include plural referents unless the context clearly dictates otherwise.

As used herein, “optional” or “optionally” means that the subsequently described event or circumstance does or does not occur, and that the description includes instances where said event or circumstance occurs and instances where it does not.

As used herein, the term “delivery vehicle” refers to any type of method suitable for transecting cells with oligonucleotides. In some embodiments, the carrier is selected from: N-acetylgalactosamine (GalNAc) for cells expressing the GalNAc receptor, aptamer A10 for cells expressing the PSMA receptor, or folic acids for cells expressing the folate receptor. According to some embodiments, the delivery vehicle is cholesterol, a cationic polymer, a liposome, a nanoparticle or any other suitable carrier. Each possibility is a separate embodiment.

### DETAILED DESCRIPTION OF THE INVENTION

According to some embodiments, disclosed herein is a method for rational design of long-acting oligonucleotides. The method employs deep learning AI trained with massive amounts of data to precisely identify chemical modifications profiles to optimize the duration of effect of oligonucleotide molecules.

According to some embodiments, the method employs deep learning AI trained with a massive amount of data-generated using combinatorial chemistry approach and large-scale functional screens-to identify chemical modifications profiles to optimize the duration of effect of oligonucleotide molecules.

According to some embodiments, disclosed herein is a method for rational design of long-acting oligonucleotides.

According to some exemplified embodiments, the method is utilized for the rational design of long-acting siRNAs or cell-entering siRNAs.

According to some embodiments, the method employs cost-effective combinatorial chemistry that can produce about 10⁶, about 10⁶ or about 10⁷ types of chemically modified oligonucleotide molecules. Each possibility is a separate embodiment.

According to some embodiments, the power of the combinatorial chemistry of this method stems from split-pool chemistry using solid support beads.

According to some embodiments, each batch produces ˜10⁶ beads, with each bead harboring chemically modified oligonucleotide molecules and a DNA barcode encoding the bead's unique chemical modifications.

According to some embodiments, the beads are screened in an innovative massively parallel functional assay to score their long-term efficacy.

According to some embodiments, the assay transforms the task of measuring function efficacy into a deep sequencing problem, allowing to collect massive amounts of data in a cost-effective manner.

According to some embodiments, these massive datasets are used to train a deep learning framework.

According to some embodiments, ˜one million data points or more (e.g 2 million, 5 million, 10 million or 100,000 million) are generated in a series of TeraBead batches to train the network with various types of chemical modifications, both well-established ones and new.

According to some embodiments, the end result is a generative model to predict the optimal combination of chemical modifications for an siRNA or other therapeutic RNAs to maximize its duration of effect.

According to some embodiments, to ensure clinical-relevance, the predictions are validated using in-vitro assays, organoids, and in-vivo models for lungs, an organ with a grave unmet need, yet paucity of targeted RNAi therapies.

According to some embodiments, the lung delivery will be performed using delivery vehicle.

Reference is now made to FIG. 1A and FIG. 1B which schematically illustrate the DeepSii platform by showing the workflow for evaluating chemical modifications of oligonucleotide molecules (Exemplified herein as siRNAs). The platform brings together a combinatorial chemistry design for simultaneous on-bead-synthesis of large collection of modified oligonucleotide molecules and corresponding DNA barcodes encoding their chemical pattern, with a high-throughput design of a functional assay for screening and evaluating the function of modified oligonucleotide molecules (such as RNAi molecules), followed by sorting of cells or beads based on the function, and further optionally with a deep learning artificial intelligence (AI)-trained for further analyses of the massive amount of data and prediction regarding the optimal durability, efficacy, and toxicity of these potential therapeutic molecules. According to some embodiments, the platform is utilized for the rational design of long-acting siRNAs.

According to some embodiments, there is provided a method (also referred to herein as “DeepSii” and “DeepSii platform”) for evaluation of chemical modifications of oligonucleotide molecules.

According to some embodiments, the evaluation of chemical modifications of oligonucleotide molecules includes screening a plurality of beads, each of the plurality of beads comprising an oligonucleotide with a unique chemical modification profile, in a massively parallel functional assay that scores the long-term efficacy of the chemical modifications of the oligonucleotide molecules.

In some exemplified embodiments, the functional assay is designed for large scale screening of chemical modifications that can improve the various properties of nucleic acid molecules, such as, for example, the durability of siRNA molecules.

According to some embodiments, the method may include one or more of the following steps (outlined in FIG. 1A):
1. First, a plurality of beads is obtained, each of the plurality of beads includes a first oligonucleotide molecule and a second oligonucleotide molecule, wherein the nucleotide sequence of the first oligonucleotide of each of the plurality of beads is identical, while its chemical modification pattern differs between different beads. Preferably, at least the first oligonucleotide is attached to the bead via a cleavable linker.
2. Second, cells are grown on the beads.
3. Third, the first oligonucleotide molecule is released from the bead, thereby allowing its uptake by the cells.
4. Fourth, the beads are sorted based on functional readouts.
5. Fifth, the sequence of the second oligonucleotide molecule on the sorted bead is determined.
6. Sixth, the chemical modification pattern of the first oligonucleotide is decoded based on the determined sequence of the second oligonucleotide molecule.
7. Seventh, the unique chemical modification pattern of the first oligonucleotide is associated with its corresponding functional readouts. Optionally the nucleotide may further be evaluated in various biological and/or clinical assays.

According to other embodiments, the method may include one or more of the following steps (outlined in FIG. 1B):
1. First, a plurality of beads is obtained, each of the plurality of beads includes a first oligonucleotide molecule and a second oligonucleotide molecule, wherein the nucleotide sequence of the first oligonucleotide of each of the plurality of beads is identical, while its chemical modification pattern differs between different beads. Preferably, at least the first oligonucleotide is attached to the bead via a cleavable linker.
2. Second, the beads are compartmentalized with a single or several cells into a nano compartment, preferably such that each nano compartment includes no more than a single bead and a single or a few cells. According to some embodiments, the single nano compartment is a 3D scaffold. According to some embodiments, the single nano compartment is a hydrogel encapsulating the bead/cell. According to some embodiments, the hydrogel is in the form of agarose droplets. According to some embodiments, the compartmentalization may be formed using cell capture techniques, such as but not limited to droplet microfluidics using microfluidic devices.
3. Third, at least the first oligonucleotide molecule is released from the bead into the compartment, thereby allowing its uptake by the cell. The second oligonucleotide may also be released in which case the compartment may be disintegrated and/or the cells recovered from the compartment. Alternatively, the oligonucleotide remains attached to the bead, in which case the compartment is maintained intact.
4. Fourth, the cells are sorted based on functional readouts. It is understood that if the second nucleotide remains attached to the beads, the sorting is conducted on the compartments (as shown in FIG. 1b), while if both nucleotides are released and taken up by the cells, the sorting may be conducted on the cells after their release/recovery from the compartment (e.g. by dissolvement or disintegration of the compartment.
5. Fifth, the sequence of the second oligonucleotide molecules of the sorted cell is determined.
6. Sixth, the chemical modification pattern of the first oligonucleotide is decoded based on the determined sequence of the second oligonucleotide molecule.
7. Seventh, the unique chemical modification pattern of the first oligonucleotide is associated with its corresponding functional readouts. Optionally the nucleotide may further be evaluated in various biological and/or clinical assays.

In some embodiment, functional readouts are sequentially sampled by sorting or repetitively sorting the cell for example at different time points after rescuing them from the droplets, thereby enabling analysis of, for example, the duration of effect of the function of the first oligonucleotide as can be deduced based on activity/expression of the reporter gene (i.e., as indicated by the reporter gene). In some embodiments, the sorting or the repetitive sorting can be of the droplets themselves. In accordance, the duration of effect of the first oligonucleotide is indicative of the long-term efficacy that a unique pattern of chemical modifications exerts on the first oligonucleotide function as designated by the functional assay. Without being bound to theory, according to some embodiments, improved duration of effect is attributed to increased durability/stability/activity of the first oligonucleotide.

As used hereinabove, the term “durability” is related to the stability of the first oligonucleotide molecule.

In some embodiments, the stability of an oligonucleotide molecule is an attribute related to the ability to resist nucleases and cellular degradation. In accordance, in some embodiments chemical modification of oligonucleotides can confer increased resistance against nucleases.

In some embodiments, the stability of an oligonucleotide molecule is an attribute related to the ability to resist autohydrolysis due to self-cleavage. In accordance, in some embodiments chemical modification of oligonucleotides can confer increased resistance against autohydrolysis due to self-cleavage.

In some embodiments, the stability of an oligonucleotide molecule is an attribute related to the half-life of the molecule. In accordance, in some embodiments chemical modification of oligonucleotides can confer increased half-life.

In some embodiment, functional readouts are sequentially sampled by sorting the cell-covered beads (for example, by measuring the fluorescence of FACS-sorted beads) to assess toxicity to the cells, which can be deduced by sorting the cells based on a reporter that is related to apoptosis such as Annexin V. In accordance, the toxicity of the first oligonucleotide is indicative of the toxicity of the chemical modifications exerts on the first oligonucleotide function as designated by the functional assay.

In some embodiments, cells or droplets are sorted based on functional readouts wherein said functional readouts refer to the measurement of the first oligonucleotide with said sorted cells or sorted droplets using sequencing technology such as DNA sequencing, RNA sequencing, or single-cell sequencing. For instance, RNA expression of reporter genes of individual sorted cells can be quantified by RNA-seq, wherein the sequence of the first and or second oligonucleotide is recorded simultaneously thereby allowing the assignment of reporter gene expression levels for cells compartmentalized with beads to the unique chemical modification pattern.

In some embodiments, the second oligonucleotide further comprises primer annealing sites. According to some embodiment, said annealing sites allow the annealing to further oligonucleotides useful for identification of the sequence of the second oligonucleotide. In some embodiments, the second oligonucleotide molecule further comprises PCR primers annealing sites. Said annealing sites may allow the amplification of the second oligonucleotide by means known in the art such as PCR.

In another embodiment, cells are sorted based on functional readouts. For instance, reporter gene expression of cells grown on beads functionalized with oligonucleotides according to the invention may be recorded individually followed by sorting cells based on reporter gene expression and recoding of the sequence of the second oligonucleotide thereby allowing the association of the unique chemical modification pattern of the first oligonucleotide with the expression levels of the reporter gene.

According to some embodiments, there is provided a method enabling evaluation of chemical modifications of oligonucleotide molecules, the method includes the step of obtaining a plurality of beads each plurality of beads includes a first oligonucleotide molecule and a second oligonucleotide molecule.

In some exemplified embodiments, the first oligonucleotide of each of the plurality of beads is chemically modified and the second oligonucleotide of each of the plurality of beads serves as a barcode that is indicative of the modification pattern of the first oligonucleotide, and therefore indicative of the beads' unique pattern of chemical modifications.

In some embodiments, the first oligonucleotide molecule of each of the plurality of beads has the same sequence with a different chemical modification profile.

In some exemplified embodiments, the first chemically modified oligonucleotide is an RNA molecule and the second barcode oligonucleotide is a DNA molecule.

In some embodiments, the first chemically modified oligonucleotide is an LNA molecule and the second barcode oligonucleotide is a DNA molecule.

In some embodiments, the first chemically modified oligonucleotide is a DNA molecule, and the second barcode oligonucleotide is a DNA molecule (e.g. a DNAnzyme).

In some exemplified embodiments, the first oligonucleotide is a therapeutic nucleic acid. Non-limited examples of therapeutic nucleic acids include: siRNAs, single-strand siRNAs, miRNAs, ASOs (antisense oligonucleotides), guide RNAs for CRISPR/Cas9, guide RNA for ADAR (adenosine deaminase acting on RNA), mRNA for expression of therapeutic peptides, and/or aptamers. Each possibility is a separate embodiment.

In some exemplified embodiments, the first oligonucleotide is siRNAs. In some embodiments, the first oligonucleotide is miRNAs. In some embodiments, the first oligonucleotide is ASOs (antisense oligonucleotides). In some embodiments, the first oligonucleotide is a single-strand siRNA. In some embodiments, the first oligonucleotide is guide RNA for CRISPR/Cas9. In some embodiments, the first oligonucleotide is aptamers.

In some embodiments, the first oligonucleotide is mRNAs.

According to some embodiments, the first step of obtaining a plurality of beads, wherein each plurality of beads includes a first oligonucleotide molecule and a second oligonucleotide molecule, is achieved by using the beads as solid support for simultaneous chemical synthesis of the first and the second oligonucleotide molecules by utilizing two orthogonal chemistries.

In some exemplified embodiments, the simultaneous chemical synthesis of oligonucleotides utilizing two orthogonal chemistries is the Acid-Base synthesis (AB-synth) that employs RNA and DNA phosphoramidites, with standard Dimethoxytrityl (DMT), which is acid-labile, and 9-fluorenylmethoxycarbonyl (Fmoc) and/or which is base-labile, as protective groups. In some exemplified embodiments, the simultaneous chemical synthesis of oligonucleotides utilizing two orthogonal chemistries is the Acid-Base synthesis (AB-synth) that employs RNA and DNA phosphoramidites, with standard Dimethoxytrityl (DMT), which is acid-labile, and Levulinyl (Lev), which is base-labile, as protective groups.

Accordingly, reference is made to FIG. 2. illustrating an exemplary step of the basic steps of Acid-Base Synthesis for the simultaneous synthesis of first oligonucleotide molecules (exemplified as siRNA molecules) and second oligonucleotide molecules (exemplified as DNA barcodes).

Optionally, the method may further include a synthesis step of adding a reporter/marker to the synthesized oligonucleotide, such as Cy3. Such step may be particularly relevant for evaluating successful entry of the oligonucleotide into the target cells, quantifying the yield of the synthesis on the bead, or the rate of release from the beads.

According to some exemplified embodiments, large scale evaluation of chemical modifications of oligonucleotide molecules is enabled due to simultaneous generation of massive amount of chemically modified first oligonucleotide and second barcode oligonucleotide, by utilizing a combinatorial chemistry approach that implements a split-pool process into the AB-synth, using beads as solid support for the synthesis. In accordance, as used herein, the term “combinatorial chemistry” is related to two processes: the split-pool for oligonucleotide synthesis on beads and DNA-encoded chemical libraries (DEL). Accordingly, reference is made to FIG. 3 illustrating two exemplary cycles of the split-pool process utilized by DeepSii. According to this exemplary embodiment, the split-pool process is utilized to synthesize the first two nucleotides of the first oligonucleotide molecules (exemplified as siRNA molecule) with a random combination of chemical modifications (exemplified as 2′-F and 2′-OMe), and the second oligonucleotide molecules (exemplified as DNA barcodes). According to some embodiments, the process includes three types of inputs: (i) a plurality of beads (e.g. about 106); (ii) the oligonucleotide sequence of interest; and (iii) the chemical modification for evaluation. According to some embodiments, the process can accommodate random combination of more than two chemical modifications (i.e., two sub-pools). According to some embodiments, each batch of synthesis will produce a plurality of beads (e.g. about 106) harboring chemically modified first oligonucleotide molecules and a second DNA barcoded oligonucleotide molecule encoding the bead's unique chemical modifications. According to some embodiments, only certain synthesis rounds will undergo split-pool, while the other will undergo regular synthesis with only one type of nucleotide.

According to some exemplified embodiments, the plurality of beads obtained in the first step of the method, are TeraBeads (Topologically Encoded RNAi Active Beads). As used herein, the term TeraBead is directed to beads harboring chemically modified siRNA molecules or single-strand siRNA molecules as the first oligonucleotide and DNA barcode encoding the bead's unique chemical modification profile as the second oligonucleotide.

In accordance, in some exemplified embodiments TeraBeads are generated using the hereinabove described combinatorial chemistry approach that implements a split-pool process into the AB-synth using beads as solid support.

According to some exemplified embodiments, the first oligonucleotide in a TeraBead is synthesized as a single-stranded siRNA with a hairpin that folds on itself.

In some embodiments, the hairpin that folds on itself creates a structure in which the passenger strand resides closer to the 5′ end and the guide strand resides closer to the 3′ end. According to some embodiments, these hairpin structures are loaded into the RNAi machinery similarly to double stranded siRNAs. According to some further embodiments, in this hairpin structure the guide-passenger duplexes are easily formed prior to cellular transfection and may be cleaved by Dicer and loaded to Ago2.

In some exemplified embodiments the first oligonucleotide is a single-stranded siRNA with a hairpin that folds on itself.

In some exemplified embodiments the first oligonucleotide is a double-stranded siRNA, such that the first strand was built using the split pool process and the other strand is identical among all beads and was added post-synthesis to the beads.

In some exemplified embodiments the bead is a TeraBead and the first oligonucleotide is a single-stranded siRNA.

According to some embodiments, the method for evaluation of chemical modifications of oligonucleotide molecules, includes the step of releasing the first oligonucleotide molecule from the beads, thereby allowing its uptake by the cells.

In some exemplified embodiments, the first oligonucleotide molecule is attached to the beads via a cleavable linker.

In some embodiments, the linker connects the first oligonucleotide to the bead via its 3′-end.

In some embodiments, the cleavable linker is a photocleavable linker.

In some exemplified embodiments, the photocleavable linker is reactive to 365 nm irradiation and creates 3′-OH at the end of the oligonucleotide.

In some exemplified embodiments, the bead is a TeraBead and the guide's strand 3′-end is connected to the bead via a photocleavable linker which allows a controllable release of the siRNA molecules from the Terabead while retaining a proper 3′-OH end, which is important for its activity.

In some embodiments, the cleavable linker is a chemically reactive linker or a biologically reactive linker. Each possibility is an embodiment.

In some embodiments, the 5′-end of the first oligonucleotide of all beads is conjugated to a small molecule such as Vitamin E, GalNac, or another small molecule that enables cellular entry using receptor mediated endocytosis. In some exemplified embodiments, the bead is a Terabead and the passenger strand's 5′-end of the siRNA is conjugated to a small molecule such as Vitamin E, GalNac, or another small molecule that enables cellular entry using receptor mediated endocytosis.

Reference is now made to FIGS. 4A-B illustrating a schematic representation of the TeraBead comprising the different components hereinabove described (i.e., hairpin siRNA, photocleavable linker, small molecule, and DNA barcode).

According to some embodiments, the method for evaluation of chemical modifications of oligonucleotide molecules, includes the step of growing cells on beads.

According to some embodiments, the cell is a mammalian cell. In some embodiments, the cells are primary cells. In some embodiments, the cells are tissue culture cells. In some embodiments, the cells are iPS-derived cells. According to some embodiments, the cells are common cell lines. According to some embodiments, the cell is a lung-derived cell. In some embodiments, the cells are cells that grow in monolayer.

According to some embodiments, the beads serve as microcarriers, and cells with a reporter gene for the function of the first oligonucleotide are grown directly on the beads. In some exemplified embodiments, the cells are subject to contact inhibition to stop cell renewal that can reduce the ability to stop their proliferation. Once the colonies on the beads are formed and stable, the first oligonucleotide is released from the beads to the attached cells using a reaction that cleaves the linker.

In some exemplified embodiments, the proliferation of the cells is controllable.

In accordance, in some embodiments the beads are TeraBeads, the cells are cells subjected to contact inhibition, the first oligonucleotide is a single-stranded siRNA with a hairpin that folds on itself, and the reaction that cleaves the linker is a photocleavage reaction.

According to some embodiments, the method for evaluation of chemical modifications of oligonucleotide molecules, further includes the step of sorting beads.

According to some exemplified embodiments, the cell-covered beads are sorted based on functional readouts provided by the activity of the reporter gene.

In some embodiments, the cell-covered beads are sorted based on fluorescence levels.

In some exemplified embodiments, the cell-covered beads are FACS-sorted based on fluorescence levels.

Reference is now made to FIG. 5A-FIG. 5B which show light micrographs of growing Hela cells attached to NittoPhase and Toyopearl beads, respectively. According to some embodiments, the beads shown in FIGS. 5A-B—(a) are amenable to nucleic acid synthesis; (b) are small enough to be sorted using flow cytometry; (c) can support cell attachment and doubling; and (d) have low autofluorescence properties.

According to some exemplified embodiments, the beads are Toyopearl and NittoPhase beads that are susceptible to synthesis of nucleic acids and sorting on cell sorter. According to some exemplified embodiments, the cells attached to the beads are common cell lines (for example, Hela, HepG2, or HEK293 cells). According to some exemplified embodiments, the FACS-sorted beads show low autofluorescence.

Reference is made to FIG. 6 that shows fluorescent-activated cell sorting (FACS) analysis of sorted Toyopearl beads demonstrating low autofluorescence properties of the beads.

According to some embodiments, the method for evaluation of chemical modifications of oligonucleotide molecules may further include the step of determining the sequence of the second oligonucleotide molecule on the sorted bead.

According to some embodiment, deep sequencing is performed to the DNA barcodes of sorted beads to determine the sequence of the second oligonucleotide molecule.

According to some embodiment, the process of sorting beads and deep sequencing of beads may be repeated at different time points after transfection, thereby collecting data on chemically modified first oligonucleotide molecules function over time, to identify determinants affecting the duration of effect of the nucleic acid molecule.

According to some embodiments, the cells and beads are co-compartmentalized into nano compartments. In a preferred embodiment, cells and beads are co-compartmentalized into droplets for example by using droplet microfluidics, which advantageously allow thousands of reproducible, compartmentalized reactions to be carried out on single beads in parallel.

According to some embodiments, the droplets may be formed in fluorocarbon oil optionally containing a surfactant that stabilizes the droplet.

According to some embodiments, droplets are made of a liquid, solution, emulsion, foam, gel, suspension and/or hydrogel. In some embodiments, droplets are made of a polymer network such as collagen, gelatin, and/or agarose droplets. According to some embodiments, droplets are agarose, gelatin and/or collagen droplets.

According to some embodiments, droplets have a volume of about 1 fL to 10 mL, preferably 1 pL to 10 μL, more preferably 10 pL to 1 μL, more preferably 100 pL to 100 nL, more preferably 50 pL to 10 nL and most preferably 100 pL to 1 nL.

According to some embodiments, droplets are microdroplets. According to some embodiments, cells and beads may be encapsulated in hydrogel microdroplets. Hydrogel droplets, such as those made of agarose, are picolitre-volume spherical scaffolds advantageously remain stable in aqueous solutions. Advantageously, since the hydrogel allows diffusion of nutrients and dissolved gases to circulate and reach the encased cells, agarose encapsulation allows cells to be grown in individual microenvironments for extended periods of time. Moreover, while conventional plate-based cell culture methods attempt to simulate a cell-growth environment in a two-dimensional plane, encapsulating cells within hydrogel droplets allows the cells to be grown in a three-dimensional scaffold that more closely mimicking their native physiological environment. According to some embodiments, the cells are encapsulated in the hydrogel droplets using droplet microfluids, as schematically illustrated in FIG. 7A.

Specifically, the droplets may be formed by utilizing a chip which allows flow of the beads through a first channel and flow of cells through a second channel, the flow being such that each droplet that is formed contained no more than one bead at a junction between the channels and zero to multiple cells, as illustrated in FIG. 7A. Furthermore, at the junction the cell and bead encounter flow flown though to oppositely positioned channels, which cause encapsulation of the cells and bead, which then, due to flow dynamics are forces to exit through a fifth channel (see FIG. 7A).

According to some embodiments, the method for evaluation of chemical modifications of oligonucleotide molecules may further include the step of decoding the chemical modification pattern of the first oligonucleotide based on the determined sequence of the second oligonucleotide molecule.

According to some embodiment, the sequencing data is used to decode the DNA barcodes of sorted beads into their corresponding patterns of first oligonucleotide chemical modifications.

Reference is now made to FIG. 8 that illustrates an exemplary process of encoding and decoding (exemplified as five rounds of split-pool with two sub-pools). According to some embodiments, the DNA barcode on each bead encodes the split-pool path that the bead underwent in a format that is accessible to high throughput sequencing. According to some embodiments, since the synthesis scheme is known, the first oligonucleotide modification of the bead can be decoded back by sequencing only the DNA barcode.

According to some embodiments, the method for evaluation of chemical modifications of oligonucleotide molecules may further include the step of associating the unique chemical modification pattern of the first oligonucleotide with its functional readouts and duration of effect.

According to some embodiments, the massive amount of generated data is leveraged to train a flexible deep learning model to learn the structure-activity relationships between the profile of chemical modification of the first oligonucleotide molecule and its function.

In accordance, according to some embodiments, the model can receive two types of inputs: (i) the chemical combination, defined as the modification type for each nucleotide in the first oligonucleotide molecule; and (ii) the sequence of the first oligonucleotide molecule, which is fixed/constant in a bead batch.

According to some embodiments, the output of the model is a prediction of long-term efficacy.

According to some embodiments, the model may predict whether a certain chemical combination induces high durability.

According to some embodiments, to predict the output from the input, deep learning models embed the input (i.e., a chemical combination and a first oligonucleotide sequence)-into a latent high dimensional space. This space in essence captures the ‘design principles’ (i.e., combination of chemical modification) that dictate the performance of the first oligonucleotide.

According to some embodiments, the method for evaluation of chemical modifications of oligonucleotide molecules may further include selecting oligonucleotides with an optimal chemical modification profile based on their functional readouts.

In some embodiments, the optimal chemical modification profile provides a sustained/desired change in the function of the oligonucleotide and/or an enhanced change in the function of the oligonucleotide, relative to a similar oligonucleotide without the optimal chemical modification profile.

According to some embodiments, the deep learning model can investigate/determine which are the most optimal modification combinations.

According to some embodiments, advantageously, the deep learning model disclosed herein, provides generative capabilities, such as feedback generative adversarial networks (GANs) that allow to efficiently explore regions in the massive search space of chemical modifications that are enriched for highly durable first oligonucleotide molecules.

According to some embodiments, the DeepSii platform can thus suggest chemical combinations for first oligonucleotides with optimized durability.

Reference is now made to FIG. 9 illustrating an exemplary deep learning framework. According to some embodiments, the deep learning process starts with a training set of chemically modified first oligonucleotides (exemplified as siRNAs) and their long-term efficacy. According to some embodiments, the set is used to train a deep learning model that accepts the first nucleic acid sequence and the chemical modifications to predict long term efficacy. Then, a generative model is used to efficiently obtain/determine/predict chemically modified optimized first oligonucleotides.

According to some embodiments, the method for evaluation of chemical modifications of oligonucleotide molecules may further include in-vivo evaluation of the duration of effect of chemically modified oligonucleotide.

According to some exemplified embodiments, the in vivo evaluation is of the duration of effect of chemically modified siRNA.

According to some exemplified embodiments, the in vivo evaluation of duration of effect of chemically modified oligonucleotide is performed in the lungs.

According to some embodiments, many respiratory diseases could benefit from genetic intervention using long-acting oligonucleotide-based therapies. In some embodiments, these diseases include, but are not limited to (1) Mendelian conditions such as Cystic Fibrosis and Primary Ciliary Dyskinesia; (2) complex traits including asthma, COPD, or pulmonary fibrosis; (3) malignancies of the lung; and (4) infectious respiratory diseases, such as SARS-CoV-2, Influenza, or RSV.

According to some embodiments, lung delivery can be done via systemic delivery.

According to some embodiments, lung delivery of chemically modified oligonucleotide can be done via inhalation.

According to some embodiments, evaluation of duration of effect of chemically modified oligonucleotide may be performed in in vitro systems that focus on modeling the target tissue.

According to some embodiments, evaluation of duration of effect of chemically modified oligonucleotide may be performed using delivery vehicle.

According to some embodiments, evaluation may be performed in human air-liquid interface organoid models of the lungs or in vivo in animals.

According to some embodiments, a back-and-forth process is performed between predictions generated by the DeepSii platform's deep learning system and validations using in-vitro and lung organoid systems.

### BRIEF DESCRIPTION OF THE FIGURES

The invention will now be described in relation to certain examples and embodiments with reference to the following illustrative figures so that it may be more fully understood.

FIG. 1A and FIG. 1B schematically illustrate the DeepSii platform workflow, for evaluating chemical modifications of oligonucleotide molecules with and with droplet encapsulation, respectively. The system brings together a combinatorial chemistry design for simultaneous synthesis of large collection of modified oligonucleotide molecules and corresponding DNA barcodes encoding their chemical pattern, on solid support beads, with a high-throughput design of a functional assay for screening and evaluating the function of modified oligonucleotide molecules (such as RNAi molecules), followed by sorting of beads based on the function, and further optionally with a deep learning artificial intelligence (AI)-trained for further analyses of the massive amount of data and prediction regarding the optimal durability of these potential therapeutic molecules, which can be then tested in-vivo.

FIG. 2 schematically illustrates the basic steps of Acid-Base Synthesis (AB-Synth) by showing simultaneous synthesize of ONT molecules and DNA barcodes using two orthogonal chemistries employing RNA and DNA phosphoramidites, with standard Dimethoxytrityl (DMT), which is acid-labile, and 9-fluorenylmethoxycarbonyl (Fmoc) which is base-labile, as protective groups.

FIG. 3 schematically illustrates DeepSii's split-pool process, exemplifying two rounds of split-pool that synthesize a 3′-UC-5′ ONT with a random combination of 2′-F and 2′-OMe. The left subpools are barcoded with DNA purine and the right subpools with pyrimidine. The process can accommodate more than two subpools as detailed in Example 1.

FIG. 4A and FIG. 4B schematically illustrate the TeraBead (FIG. 4A) and the components of the TeraBead (FIG. 4B): (1) Hairpin siRNA using a single strand (2) Photocleavable linker that is reactive to 365 nm irradiation and creates 3-OH end of the siRNA (3) A small molecule to promote receptor mediated endocytosis (4) A DNA barcode to register the bead path.

FIG. 5A and FIG. 5B shows a light micrograph of growing Hela cells attached to NittoPhase (FIG. 5A) or Toyopearl (FIG. 5B) beads.

FIG. 6 shows fluorescent-activated cell sorting (FACS) analysis of sorted Toyopearl beads. The graphs presented in FIG. 6 show the red (left column) and green (right column) channels of sorted beads (top) compared to GFP+ (middle), and mCherry+ (bottom) cells as a positive control. The beads are negative compared to the positive cells.

FIG. 7A schematically shows the process of encapsulating the cells into single compartments, each compartment also including a single bead.

FIG. 7B and FIG. 7C show microscope images of a plurality of single droplets (also referred to herein as compartments) generated using droplet microfluidics, before (FIG. 7B) and after (FIG. 7C) exposure to UV, respectively. The beads inside the droplets are covered with oligonucleotides molecules that are labeled with Cy3 and are attached via a photocleavable linker to the bead. Each droplet either includes a single bead (red fluorescence) or no bead (no florescent signal observed).

FIG. 7D shows florescence intensity of the supernatant of beads as a function of 365 nm light exposure of the beads. In blue: beads covered with oligonucleotides molecules that are labeled with Cy3 and are attached via a photocleavable linker to the bead. In orange: covered with oligonucleotides molecules that are labeled with Cy3 and permanent linker to the bead.

FIG. 8 schematically illustrates DeepSii's encoding and decoding process exemplifying: Top: five rounds of split-pool with two subpools (circles). The red line presents the path of one random bead. Bottom: The corresponding path encoded on the DNA barcode using purine addition in the left subpool and pyrimidine addition in the right sub-pool.

FIG. 9 schematically illustrates the deep learning process (framework) of the DeepSii platform. The deep learning process includes a training set of chemically modified siRNAs and their long-term efficacy. The set is used to train a deep learning model that accepts the sequence and modifications to predict long term efficacy. Then, a generative model is used to efficiently obtain chemically optimized siRNAs.

FIG. 10 left panel shows fluorescence microscopy images of cells encapsulated in microdroplets with either Alexa674- or Alex488-labeled DNA barcodes (light gray/white) following incubation. Right panel shows fluorescent-activated cell sorting (FACS) analysis of rescued cells sorted based on the DNA barcodes taken up.

FIG. 11 shows a light micrograph of growing A549 cells, which were first encapsulated in microdroplets and subsequently rescued and plated for viability tracking.

FIG. 12 shows mRNA expression levels for Huntingtin normalized to GAPDH for A 549 cells encapsulated in microdroplets in the presence of indicated siRNAs.

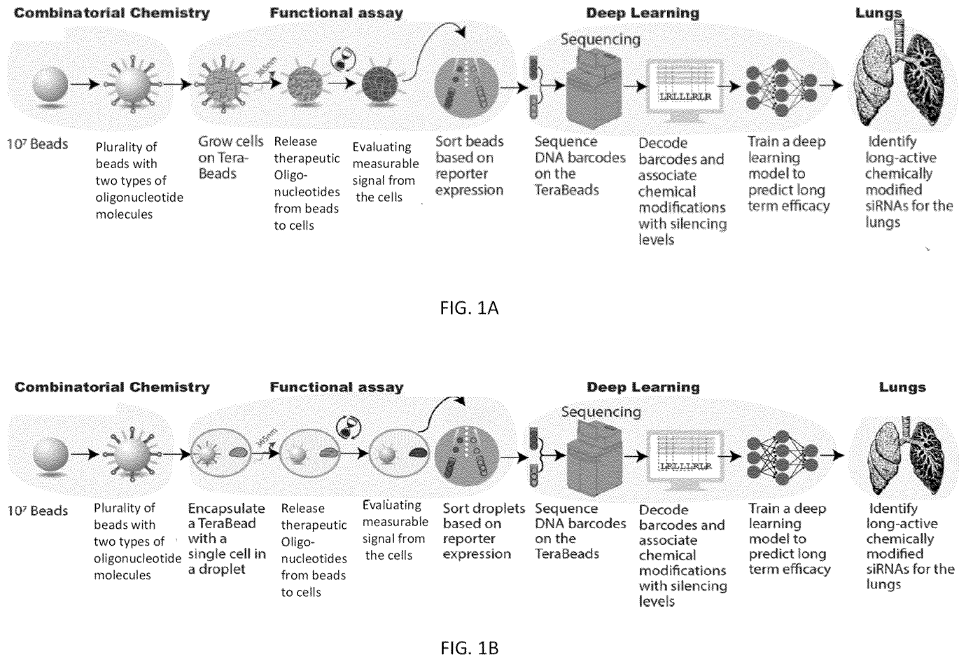

### EXAMPLES

The following examples exemplify the use of the herein disclosed DeepSii platform as a principled end-to-end framework to generate, assay, and analyze massive amounts of data in order to rationally design long lasting siRNA therapies. It is however understood by one of ordinary skill in the art that chemically modified siRNA serves as an example only, and that some of the design principles of the DeepSii platform may be applicable for other chemically modified nucleic acid therapeutics, such as ASOs, CRISPR/Cas9 gRNAs, aptamers and mRNAs.

### Example 1

### Simultaneous Generation of Massive Amounts of Chemically Modified siRNA Molecules and DNA Barcodes Using Combinatorial Chemistry

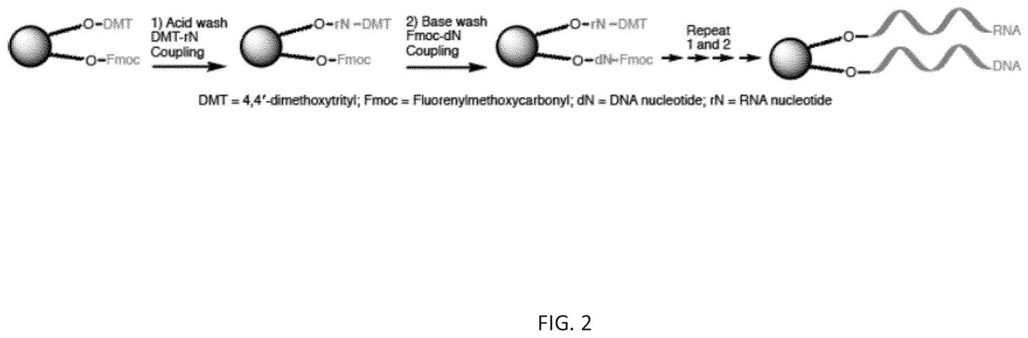

The approach disclosed herein starts by harnessing a combinatorial chemistry process that allows screening large amounts of chemically modified siRNAs. To this end, multiple rounds of split-pool synthesis using Acid-Base synthesis (AB-Synth) is employed. AB-Synth can simultaneously generate two types of oligonucleotides: the siRNA molecule and a DNA barcode. To avoid cross-talk, AB-Synth harnesses two orthogonal chemistries. For the RNA synthesis, it utilizes RNA phosphoramidites that are protected with the standard Dimethoxytrityl (DMT), which is acid-labile. For the DNA synthesis, it uses DNA phosphoramidites that are protected with a base-labile group, such as 9-fluorenylmethoxycarbonyl (Fmoc). By alternating between acid and base washes, AB-Synth can simultaneously grow the two molecules. As overview of the AB-Synth methodology is illustrated in FIG. 2.

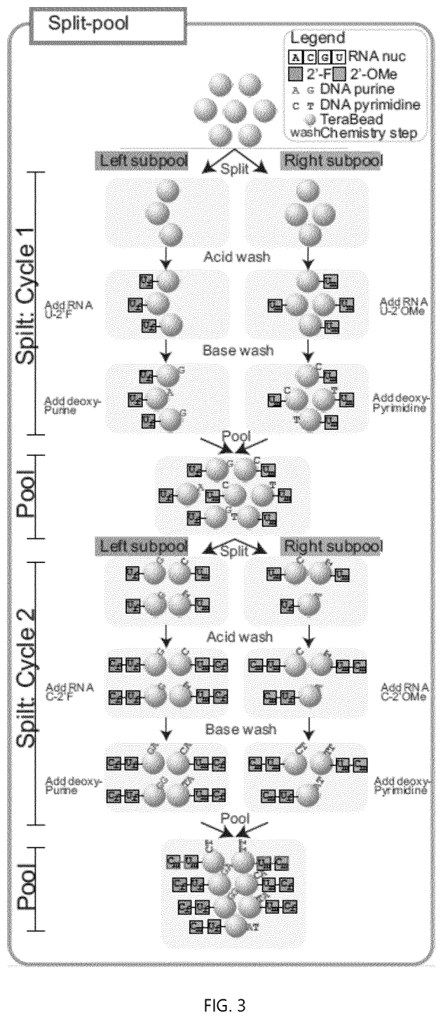

The AB-synth is orchestrated in a split-pool process (illustrated in FIG. 3), to generate the TeraBeads (Topologically Encoded RNAi Active Beads). The process starts with three types of inputs: (i) >106 beads; (ii) the siRNA sequence of interest; and (iii) the chemical modification being evaluated. For example, an siRNA molecule with a 3′-end: 3′-UCGGA-5′ and two chemical modifications: 2′-F and 2′-OMe. In the first cycle, the beads are split at random into Left and Right sub-pools and each pool is subjected to acid wash after which an RNA Uracil-DMT is attached to the beads, where the Left sub-pool receives a rU-2′-F and the Right sub-pool receives rU-2′-OMe (reminder: oligo synthesis works 3′→5′). Next, while still separated, the two sub-pools are washed with a base. A mixture of deoxy-purine-Fmoc is added to the Left sub-pool and a mixture of deoxy-pyrimidine-Fmoc is added to the right sub-pool. Finally, the two sub-pools are pooled together. In the next split-pool cycle, the beads are split again into two random sub-pools and the same exact process continues but this time with a rC-2′-F and rC-2′-OMe in the Left and Right sub-pools, respectively. Half of the beads that were on the Left sub-pool in the first cycle end up in the Right sub-pool in the second cycle and half of the beads end up in the Left sub-pool. Thus, by the end of the second cycle, equal mixtures of 3′-Uf-Cm, 3′-Uf-Cf, 3′-Um-Cm, and 3′-Um-Cf are generated. Then, the process continues similarly for the next nucleotides: G,G,A. By repeating the split-pool process for the GGA end, each bead samples a random path of sub-pools. This path creates a unique pattern of chemical modifications shared by all siRNA molecules on the same bead but not between the beads. Importantly, since the same nucleobases are used in both sub-pools in each round (e.g. U in cycle #1, C in cycle #2), all beads have the same siRNA sequence regardless of the chemical combination. This allows evaluating the effect of applying many different chemical modifications to the same siRNA sequence.

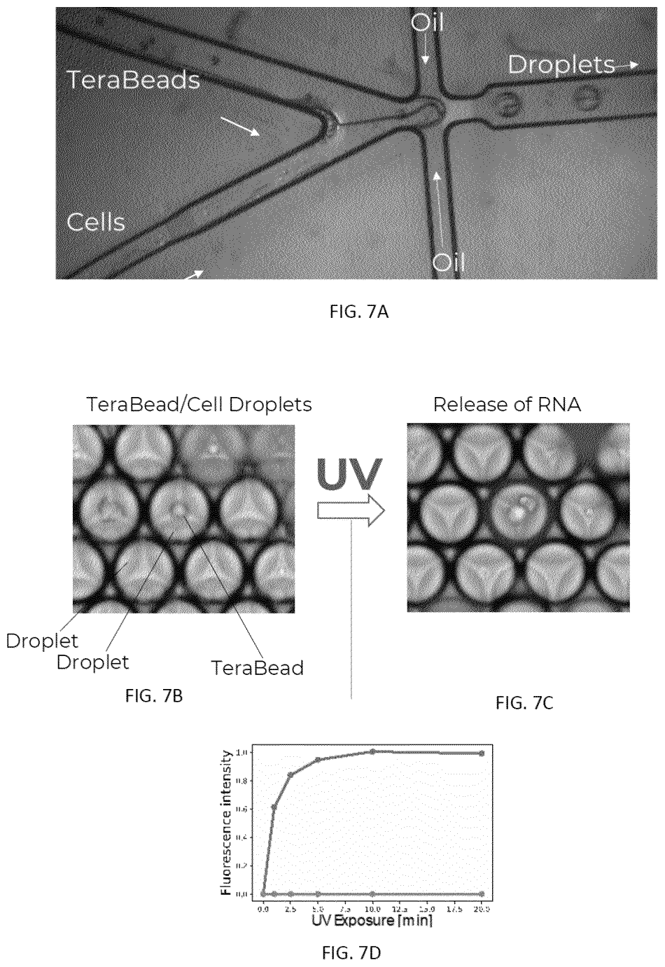

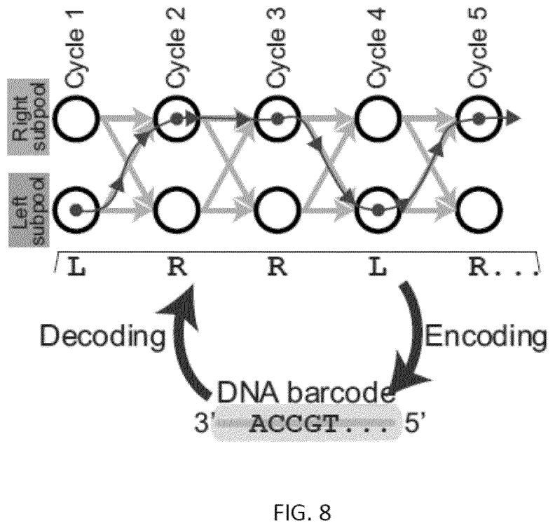

The DNA barcode on each TeraBead encodes the split-pool path that the bead underwent in a format that is accessible to high throughput sequencing (as illustrated in FIG. 7). In the example above, a purine in the i-th position from the 3′ end of the DNA barcode means that in the i-th cycle, the bead was in the Left sub-pool. Conversely, a pyrimidine means that the bead was in the right sub-pool. Since the synthesis scheme is known, the siRNA modification of the bead can be decoded back by sequencing only the DNA barcode. Hence, the TeraBead approach allows decoding highly complex chemical modification schemes via high throughput sequencing. Moreover, we can have more than one synthesis cycle of DNA in each sub-pool as a means for error-correcting code. For example, every fourth round we can add a three “T” nucleotides to the DNA oligonucleotide in all subpools that will act as a synchronization symbol to correct for indel error.

Importantly, the herein disclosed TeraBead approach creates a highly flexible combinatorial process to synthesize chemically modified siRNAs. The example above, considers only 2′-F and 2′-OMe. However, a wide range of RNA modifications can be utilized, including combinatorial changes to the phosphate group (e.g. phosphorothioate), to the base (e.g. N6-methyladenosine), or to the sugar (e.g. 2′-M-OE), or combinatorial changes to a small molecule at each end of the oligo (e.g. GalNac) and different modifications can be applied in different cycles. Moreover, the process is not limited to two sub-pools as in the example above. The number of sub-pools can be the number of ports of the oligonucleotide synthesis machine and the practical number of DNA encoding steps to represent all sub-pools (more than 4 sub-pools can be encoded by performing two base washes in every split). In addition, only some of the positions of the siRNA can be varied but not others by switching from a split-pool to a regular column synthesis. With this flexibility, it is possible to focus the large number of data points to the most promising areas of the chemical search space. This property is used extensively in the construction of the AI model.

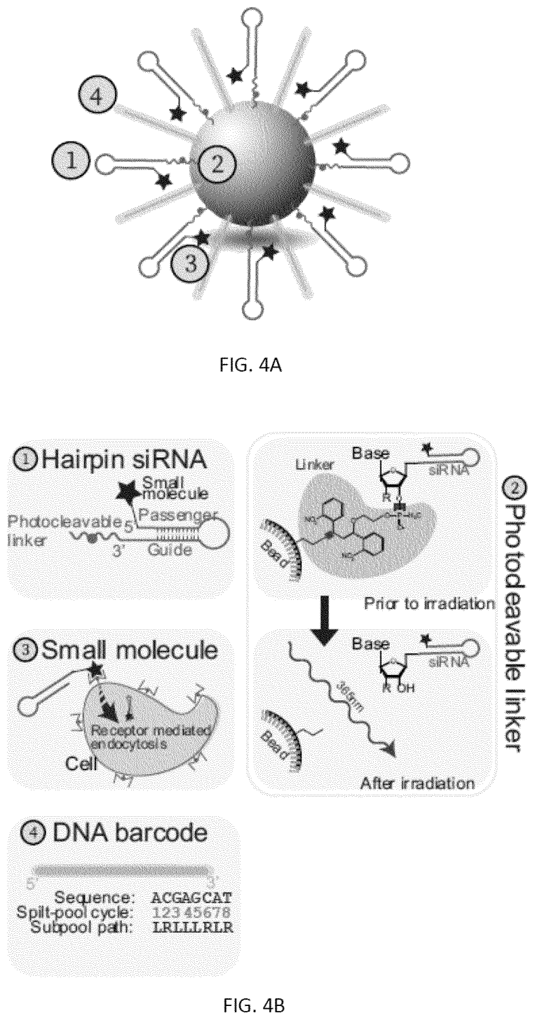

The siRNAs on the TeraBeads have several additional properties that are important for their function. First, each siRNA is synthesized as a single stranded molecule with or without a hairpin that folds on itself. In case of this hairpin structure, the passenger resides closer to the 5′ end and the guide resides closer to the 3′ end. These hairpin structures are loaded into the RNAi machinery similarly to double stranded siRNAs. The advantage of this structure is that the guide-passenger duplexes are easily formed prior to cellular transfection and enabling loading to the RNAi machinery. Alternatively, a single-strand siRNA can be synthesized and used instead. Second, the guide's 3′-end is connected to the bead via a photocleavable linker that is sensitive to UVA irradiation. This allows a controllable release of the siRNA molecules from the bead, while retaining a proper 3′-OH end, which is important for its activity. Finally, the 5′-end of the siRNA can be conjugated to a small molecule such as Vitamin E, GalNac, or another small molecule that enables cellular entry using receptor mediated endocytosis. FIGS. 4A-4B show schematic illustration of Terabeads and siRNA molecules associated therewith.

Thus, the combinatorial approach disclosed herein breaks new grounds in the synthesis scale of modified siRNAs. Certain screening methods for chemically-modified siRNAs rely on low-throughput column-based synthesis platforms that typically produce up to ˜192 siRNAs/day. Conversely, the combinatorial approach disclosed herein generates a large plurality (about 10⁶) TeraBeads in one batch, Accordingly, the disclosed synthesis method exhibits 3 orders of magnitude greater throughput and 3-4 orders of magnitude greater cost-effectiveness than current approaches.

### Example 2

### A High Throughput Method to Functionally Assess the Long-Term Efficacy of siRNAs

Once the chemically modified siRNAs are generated, there is a need to extract a readout regarding their long-term efficacy. Current methods typically rely on reporter assays to obtain these functional readouts. In a typical setting, an siRNA is introduced into a single well with cells containing a fluorescent gene (e.g. GFP) with a target site in its 3′UTR that matches the siRNA. Next, the wells are imaged by a plate reader to quantify siRNA silencing. However, standard reporter assays scale poorly. Tens of thousands of plates would be needed for a library with 10⁶ RNAi triggers, rendering the use of reporter assays impractical in the herein disclosed setting.

Accordingly, a massively parallel method to extract functional readouts is utilized herein. In the method disclosed herein, the TeraBeads serve as microcarriers, and cells with a reporter gene and a matching target site are grown directly on beads as monolayers. Cells that are subject to contact inhibition are specifically used to stop cell renewal that can reduce the ability to stop their proliferation.

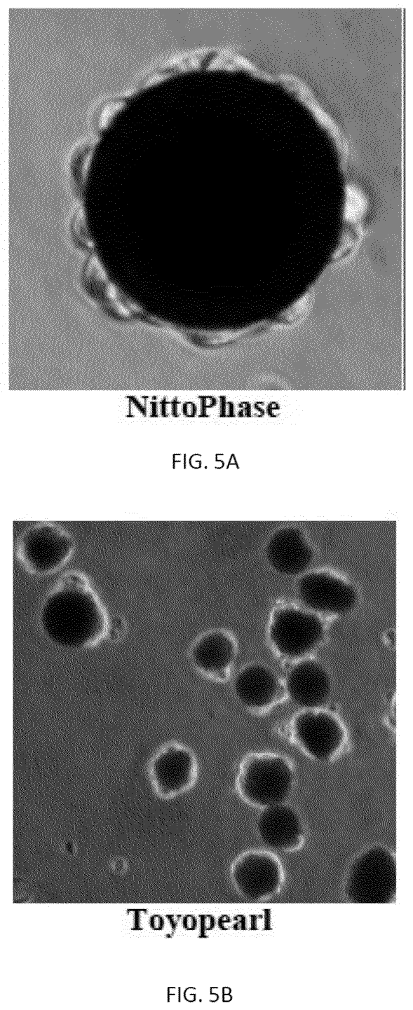

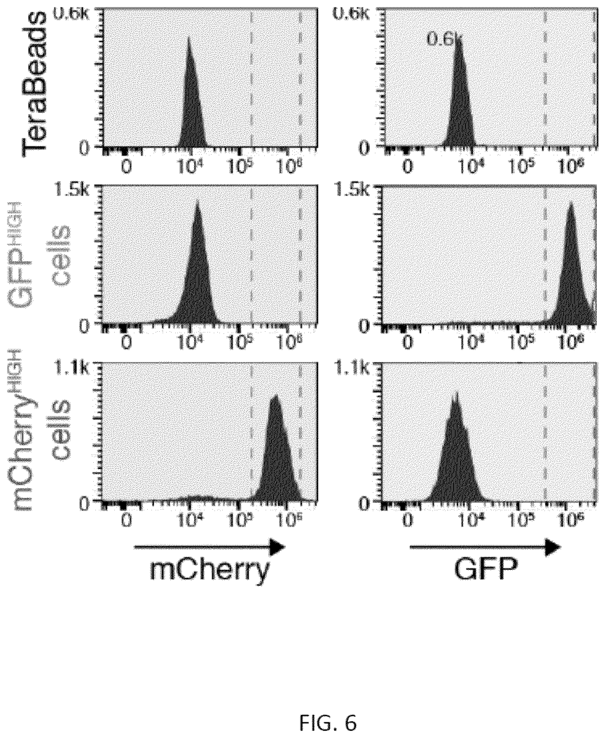

To this aim, several types of microcarrier beads were tested. The focus was on beads that are (a) amenable to nucleic acid synthesis; (b) small enough to be sorted using flow cytometry; (c) can support cell attachment and doubling; and (d) have low autofluorescence properties. Results were obtained for Toyopearl hw-65s, and for Toyopearl NH2-750F beads that were susceptible to synthesize of nucleic acids and sorting on a BD Influx cell sorter equipped with a 200 μm nozzle. Light micrographs of such beads are shown in FIG. 5 that shows the growth of Hela cells attached to NittoPhase UnyLinker 200 (10-00-03-200) (FIG. 5A) or Toyopearl NH2-750F (FIG. 5B) beads. Low autofluorescence of beads was demonstrated by fluorescent-activated cell sorting (FACS) analysis of sorted Toyopearl beads (FIG. 6) Similar results were observed with NittoPhase UnyLinker 200 and a large particle flow-cytometry (Unionbio) (data not shown) as well as for various cell lines (including, HepG2 and HEK293 cells) (data not shown).

Once the colonies on the beads are formed and stable, the siRNAs were released from the beads to the attached cells using UVA irradiation. Without being bound by any theory, the siRNAs entered the cells via receptor mediated endocytosis and attenuate the reporter gene. As each bead was covered with homogenous siRNA molecules, all the cells on the same bead received the same RNAi treatment. After a period of time to assess the siRNA duration of effect, the cell-covered beads were FACS-sorted into buckets based on fluorescence levels.

Next, deep sequencing was performed on the DNA barcodes in each bucket. In its maximum capacity, NovaSeq S4 can produce 1010 reads in a single run, providing ˜1000× sequencing coverage for every TeraBead. Finally, the sequencing data was used to decode the DNA barcodes in each bucket back into their corresponding patterns of siRNA chemical modifications. By repeating the process at different time points after transfection, data on chemically modified siRNA activity can be collected across time to identify determinants affecting the duration of effect.

The massively parallel approach disclosed herein is advantageously highly cost effective. The main costs are maintaining the microcarrier tissue culture, sorting, and high throughput sequencing. Since tens of cells are grown per bead and all the beads are pooled together, the approach substantially minimizes tissue culture costs. Sorting is also a high throughput process: Based on the results presented it is possible to sort the beads in 24 hours, further reducing costs.

### Example 3

### A High Throughput Method to Functionally Assess the Long-Term Efficacy of siRNAs Using Microfluidics

An alternative method for massive parallel extraction of functional readouts is also provided. According to this method, the TeraBeads serve as microcarriers, however instead of growing the cells with the reporter gene directly on the beads, single cell compartmentalization based on microfluids is utilized.

Using optimized run parameters, cells and TeraBeads were encapsulated in droplets using QX200™ Droplet Generation Oil for EvaGreen (#1864005, BioRad) and a Nadia Inovate droplet machine (FIG. 7A). After a run had ended, half the resultant emulsion was irradiated on ice for 10 minutes with 365 nm UVA using UVA crosslinker (UV products) in order to release the oligo from the TeraBeads into the droplet milieu. The other half was kept unirradiated on ice. As seen from FIG. 7B and FIG. 7C, droplets, bead-containing or devoid of beads, were visualized following, the encapsulation (FIG. 7B), and after the UVA irradiation (FIG. 7C). FIG. 7D shows the efficacy of oligo release from TeraBeads as a factor of UVA irradiation time (blue line). The oligo used was labelled with a Cy3 fluorophore and its release to the medium was quantified using a fluorescence microplate reader. Red line indicates control beads containing cy3 labelled oligonucleotide, but without the photocleavable linker.

### Example 4

### A Powerful Deep Learning Methodology that Predicts Long Term Efficacy

The current practice for identifying long-acting siRNAs relies on evaluating a handful of patterns with a limited set of chemical building blocks. However, the potential search space is overwhelmingly large. A given siRNA sequence can be chemically modified in >1012 different combinations, assuming only two chemical building blocks, such as 2′-F and 2′-OMe. Hence, the current approach is cumbersome and is akin to searching for a needle in a haystack. By contrast, the approach disclosed herein leverages the massive amounts of generated data to train a flexible deep learning model.

Deep learning is a powerful framework that can learn the structure-activity relationships of molecules from massive amounts of observations. The deep learning model disclosed herein receives two types of inputs: (i) the chemical combination, defined as the modification type for each nucleotide in the guide and passenger strands; and (ii) the sequence of the siRNA, which is fixed in a TeraBead batch. These two inputs are represented by the common one-hot scheme. The output of the model is a prediction of long-term efficacy. To predict the output from the input, deep learning models embed the input—a chemical combination and an siRNA sequence in the present case—into a latent high dimensional space. This space captures the ‘design principles’ that dictate siRNA performance.

Deep learning also confers a powerful framework for generating novel combinations of chemistries in a highly efficient matter. The model not only predicts whether a certain chemical combination induces high durability, but it also investigates which are the most optimal combinations. However, with a model that can only predict, a brute force approach would necessitate computationally enumerating all >1012 possible combinations to find the best ones, which is infeasible in practice. Certain deep learning models offer generative capabilities, such as feedback generative adversarial networks (GANs). These allow to efficiently explore regions in the massive search space of chemical modifications that are enriched for highly durable siRNAs. Using these frameworks, DeepSii suggests chemical combinations for siRNAs with optimized durability.

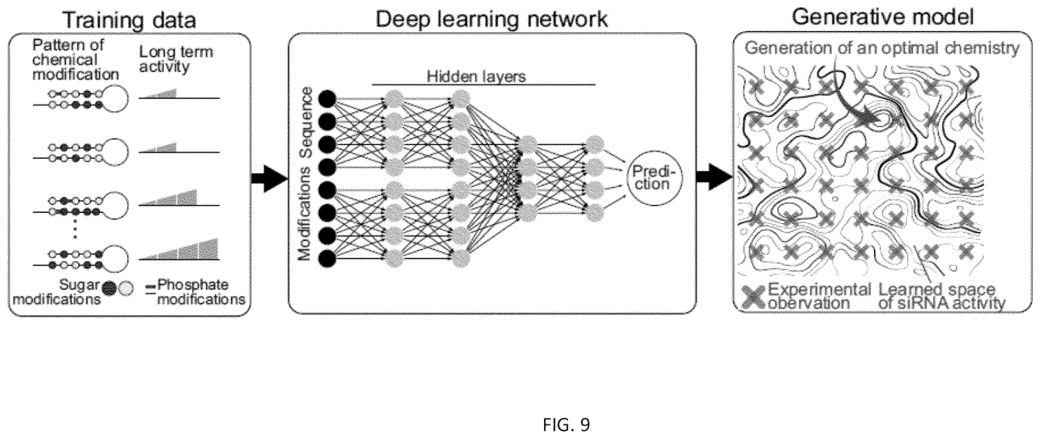

The main challenge of deep learning is that it is a data hungry process requiring massive amounts of data to realize its potential. For this reason, about 2×108 data points of siRNAs modifications are generated. These data points are being generated in batches of the split-pool process. First, the network is trained with previously established modifications, namely 2′-F, 2′-OMe, and PS, and performance is compared to previous results. Next, this is followed with a screen of 5-10 newly chemical modifications and selection of the most promising 2-3 ones for deep evaluation. FIG. 9 schematically shows various steps of the deep learning model.

### Example 5

### In-Vivo Evaluation of Chemically Modified Nucleic Acid Molecules

The DeepSii platform can be used in various tissues and clinical settings. To this aim, various cellular and animal models may be utilized.

In this Example the platform was deployed in a specific clinical purpose--increasing the duration of effect of siRNA in the lungs. Lungs were used in this example for several reasons: First, many respiratory diseases could benefit from genetic intervention using long-acting siRNA therapies. These diseases include, for example, (1) Mendelian conditions such as Cystic Fibrosis and Primary Ciliary Dyskinesia; (2) complex traits including asthma, COPD, or pulmonary fibrosis; (3) malignancies of the lung; and (4) infectious respiratory diseases, such as SARS-CoV-2, Influenza, or RSV. Second, lung delivery can be done via inhalation compared to systemic delivery as required for most organs. This route increases the relevance of in-vitro system that focuses on modeling the target tissue and not on siRNA stability in the plasma.

Accordingly, TeraBeads are tested using three lung-derived cell types. Predictions generated by the DeepSii platform's deep learning system and functional validations using in-vitro and lung organoid systems are conducted. Such results maintain AI efforts focused on clinically meaningful results. The AI system is accordingly validated and In-vivo experiments are utilized to test the chemically modified siRNAs in the lungs.

### Example 6

### Conjugate Mediated Oligonucleotide Uptake in Microfluidic Droplets

The cellular uptake of oligonucleotides according to the present invention enables functional readouts allowing the association of chemical modification patterns and sequence with biophysical properties such as stability and potency.

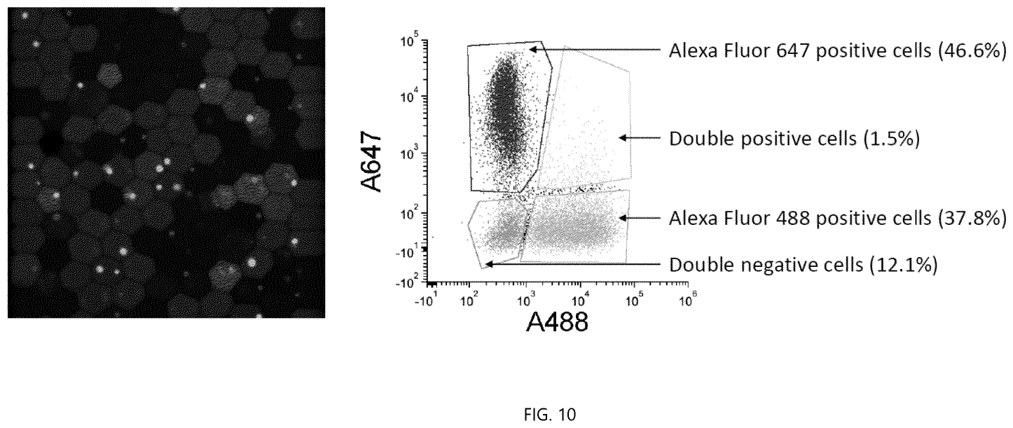

In order to test the ability of oligonucleotide uptake by cells in high-throughput approaches, microfluidics was used to generate droplets containing: (i) cells and Alexa Fluor 488 labelled DNA barcodes; and (ii) cells and Alexa Fluor 647 labelled DNA barcodes. All barcodes were conjugated to Cholesteryl-triethylene glycol (TEG) to mediate cellular uptake. Next, droplets containing DNA barcodes were mixed in a 1:1 ratio and incubated together for 24 hours at 37 degrees Celsius to allow cellular uptake of the fluorescently labelled barcodes (FIG. 10, left panel).

Following co-incubation, cells were rescued from the droplets and co-cultured for a further 24 hours before being subjected to Fluorescence activated cell sorting (FACS) analysis (FIG. 10, right panel). The analysis demonstrates uptake of Alexa Fluor 647 and Alexa Fluor 488 labeled DNA barcodes by the cells, and minimal cross-droplet contamination of DNA barcodes (double positives <2% of all cells).

### Example 7

### Viability of Cells in Microdroplets

The ability of cells to survive within droplets during microfluidics-guided encapsulation of beads and cells is critical for subsequent biological assays measuring gene expression of reporter genes or other biological properties of cells following uptake of the first oligonucleotide.

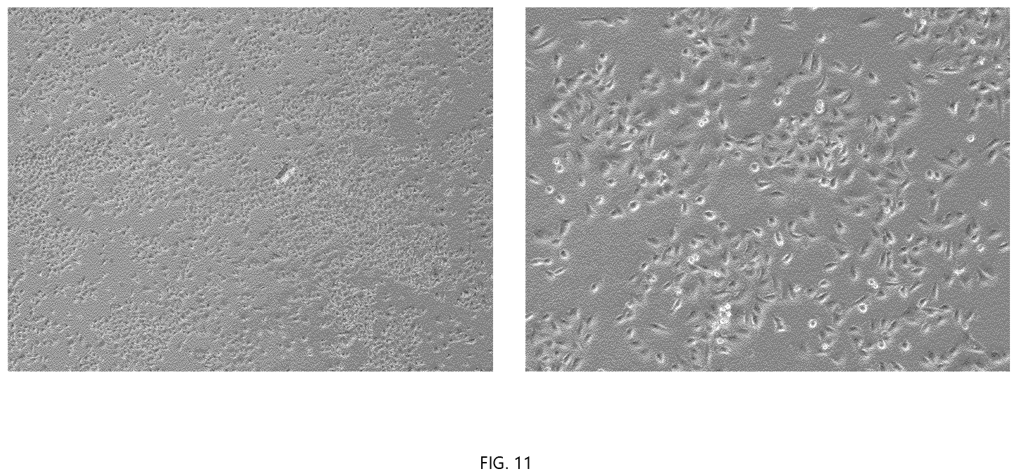

In order to assess the viability of cells during and after microfluidics-guided encapsulation, A549 cells were encapsulated in nano-liter droplets (microdroplets) using microfluidics and were incubated inside droplets for 24 hours at 37 degrees Celsius. Next, the cells were released from the droplets and plated on a tissue culture dish. Images (FIG. 11) show high viability and proliferation of the cells (left and right images, 4× and 20× magnifications respectively.

### Example 8

### Functional Readout of Oligonucleotide Properties

The ability to measure the biophysical properties of the first oligonucleotide according to the invention is critical for the association of sequence and chemical modification patterns with desired biological effects such as the knock-down efficiency of target genes exerted by said oligonucleotide.

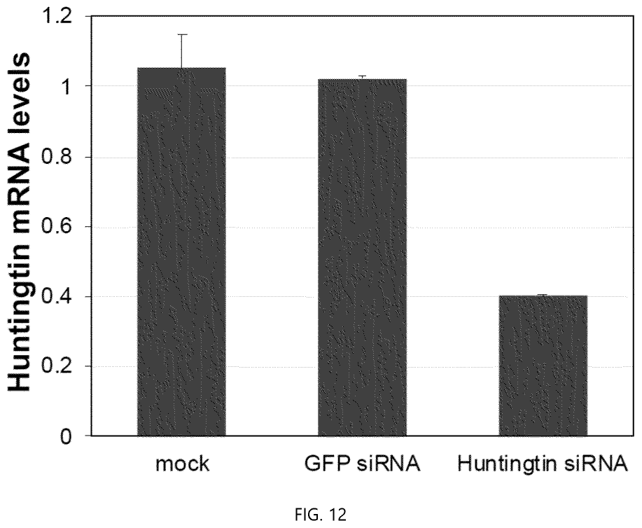

In order to show an embodiment for the measurement of biophysical properties of the first oligonucleotides according to the invention, A549 cells were encapsulated inside droplets together with either 1.5 uM non-targeting siRNA (GFP) or siRNA targeting Huntingtin. Cells were incubated inside droplets for 24 hours at 37 degrees Celsius, 5% CO2. Post-incubation, cells were released from the droplets and grown for a further 48 h in a tissue culture dish. Cells were then harvested, and RNA extracted for quantitative Real-time PCR analysis. Results shown in FIG. 12 are the relative Huntingtin mRNA levels normalized to a housekeeping gene (GAPDH). No effect can be seen for the non-targeting GFP control, while Huntingtin levels are reduced by 60% compared to the mock transfection control. These data illustrate the ability of cells encapsulated in the presence of the beads attached to oligonucleotides in microdroplets to take up said oligonucleotide, which can exert biological effects within the target cell.

While certain embodiments of the invention have been illustrated and described, it will be clear that the invention is not limited to the embodiments described herein. Numerous modifications, changes, variations, substitutions, and equivalents will be apparent to those skilled in the art without departing from the spirit and scope of the present invention as described by the claims, which follow.

What claimed is:

1. A method for evaluating chemical modifications of oligonucleotide molecules, the method comprising:
a. obtaining a plurality of beads each of the plurality of beads comprising a first oligonucleotide molecule, and a second oligonucleotide molecule,
wherein the first oligonucleotide molecule is attached to the beads via a cleavable linker,
wherein the first oligonucleotide molecule of each of the plurality of beads has a same sequence with a different chemical modification profile, and
wherein a sequence of the second oligonucleotide molecule is indicative of the chemical modification profile of the first oligonucleotide molecule,
b. juxtaposing cells to the beads, wherein the first oligonucleotide is intended for altering a biological function/activity of the cells;
c. releasing the first oligonucleotide molecule from the beads, thereby allowing its uptake by the cells;
d. sorting the cells based on functional readouts,
e. determining the sequence of the second oligonucleotide molecule on the sorted cells or beads;
f. decoding the chemical modification profile of the first oligonucleotide molecule, based on the determined sequence of the second oligonucleotide molecule; and
g. associating the chemical modification profile of the first oligonucleotide molecule with its functional readouts.

2. The method of claim 1, wherein step c. comprises releasing the first and the second oligonucleotide from the beads.

3. The method of claim 1 or 2, further comprising selecting oligonucleotides with an optimal chemical modification profile based on their functional readouts.

4. The method of claim 3, wherein said optimal chemical modification profile provides a sustained/desired change in the function of the oligonucleotide and/or an enhanced change in the function of the oligonucleotide, relative to a same oligonucleotide without the optimal chemical modification profile.

5. The method of any one of claims 1 to 4, wherein the first and the second oligonucleotide molecules are independently DNA or RNA molecules.

6. The method of any one of claims 1 to 5, wherein the first oligonucleotide molecule is a gRNA molecule for a CRISPR system, an ASO molecule, a gRNA molecule for ADAR, an mRNA, an LNA, or an aptamer.

7. The method of any one of claims 1 to 6, wherein the first oligonucleotide molecule is an siRNA molecule.

8. The method of claim 7, wherein the siRNA comprises a hairpin that folds on itself.

9. The method of claim 7, wherein the siRNA is a single stranded siRNA.

10. The method of claim 9 wherein the single stranded siRNA is annealed on beads to the complementary siRNA strand.

11. The method of any one of claims 1 to 6, wherein the first oligonucleotide molecule is an mRNA molecule.

12. The method of any one of claims 1 to 11, wherein the second oligonucleotide is DNA.

13. The method of any one of claims 1 to 12, wherein the second oligonucleotide molecule further comprises PCR primers annealing sites.

14. The method of any one of claims 1 to 13, wherein the cleavable linker is a photocleavable linker.

15. The method of any one of claims 1 to 14, further comprising a step of conjugating a small molecule to the first oligonucleotide molecule, the small molecule facilitating and/or enhancing cell entry.

16. The method of claim 15, wherein the small molecule is selected from the group of Vitamin E, Cholesterol, GalNac, Cholesterol-PEG, Cholesterol-TEG, Lithocolic-oleyl, Lauryl, Myrstoyl, Palmitoyl, Steroyl, Docosanyl, Oleoyl, Linoleoyl, or any combination thereof.

17. The method of claim 16, wherein the small molecule is Vitamin E, Cholesterol-TEG, and/or GalNac.

18. The method of any one of claims 1 to 17, wherein juxtaposing cells to the beads comprises growing cells directly on the beads.

19. The method of claim 18, wherein the proliferation of the cells is controllable.

20. The method of claim 19, wherein the cells are subject to contact inhibition.

21. The method of any one of claims 18 to 20 wherein sorting the cells comprises sorting the cells together with the beads on which they are grown.

22. The method of any one of claims 1 to 20, wherein the cells express at least one exogenous reporter gene.

23. The method of any one of claims 1 to 17, wherein juxtaposing cells to the beads comprises encapsulating the cells and the beads into single nano compartments, such that each nano compartment receives no more than one bead and at least one cell, and wherein releasing the first oligonucleotide comprises releasing it into the nano compartment.

24. The method of claim 23, wherein the nano compartment is a droplet.

25. The method of claim 24, further comprising releasing at least one first oligonucleotide molecule from the bead and into the droplet, thereby allowing its uptake by the cells.

26. The method of claim 24 or 25, wherein sorting the cells comprises recovering the cells from the droplet.

27. The method of claim 26, wherein sorting the cells comprises sorting the cells, after their recovery from the droplet.

28. The method of any one of claims 24 to 27, wherein the droplet is a microdroplet.

29. The method of any one of claims 24 to 28, wherein the droplets are agarose, gelatin and/or collagen droplets.

30. The method of any one of claims 1 to 29, further comprising applying a machine learning module, wherein the applying comprises providing a first input regarding a combination of chemical modification profile on each of the first oligonucleotide, and a second input regarding the sequence of the first oligonucleotide molecule.

31. The method of claim 30, wherein the output of the machine learning module is a combination of chemical modification associated with optimal first oligonucleotide function.

32. The method of claim 30 or 31, wherein the applying of the machine learning module comprises applying a feedback generative adversarial network.

33. A computational platform for high-throughput analysis of chemical modifications of oligonucleotides, the platform comprising a processor configured to:
a. obtaining data, obtained or obtainable according to the method of any one of claims 1 to 32, indicative of a chemical modification pattern of a plurality of beads each of the beads comprising an oligonucleotide molecule having a same sequence with a different chemical modification profile, wherein the oligonucleotide is capable of affecting the functional read-out of its biological activity;
b. obtaining data indicative of the functional read-out of the biological activity of the oligonucleotide; and
c. applying a machine learning algorithm to:
d. determine the chemical modification pattern based on the data indicative thereof; and
e. associate the chemical modification profile of the oligonucleotide molecule with the functional readout of the reporter gene.

34. The platform of claim 33, wherein the processor is further configured to identify oligonucleotide sequences having an optimal chemical modification profile based on the association.

35. The platform of claim 33, wherein the processor is further configured to apply a same or different machine learning algorithm to derive a structure-activity-relationship capable of predicting an optimal chemical modification profile of an oligonucleotide associated with one or more desired characteristics of the oligonucleotide, wherein the one or more characteristics is selected from stability, efficacy, durability, or any combination thereof.
